SEQUENCE LISTING (12 sequences)

SEQ ID NO: 1 - DNA, 20 bp, Artificial Sequence
  Synthetic
ttgctttttcccagatgagc

SEQ ID NO: 2 - DNA, 20 bp, Artificial Sequence
  Synthetic
acactccaaggccgctttac

SEQ ID NO: 3 - DNA, 34 bp, Artificial Sequence
  Synthetic
ccttgtcacacgagccagtgttagtacctacacc

SEQ ID NO: 4 - DNA, 32 bp, Artificial Sequence
  Synthetic
tatttttattataaggcctgctgaaaatgact

SEQ ID NO: 5 - DNA, 27 bp, Artificial Sequence
  Synthetic
gaattagctgtatcgtcaaggcactct

SEQ ID NO: 6 - DNA, 34 bp, Artificial Sequence
  Synthetic
ccacaagtttatattcagtcattttcagcaggcc

SEQ ID NO: 7 - DNA, 20 bp, Artificial Sequence
  Synthetic
ctgcgcaagccaggcatgaa

SEQ ID NO: 8 - DNA, 20 bp, Artificial Sequence
  Synthetic
gagccctcccgactctgcga

SEQ ID NO: 9 - DNA, 33 bp, Artificial Sequence
  Synthetic
tcgcagtaagagacgcaagtctcagcattcatg

SEQ ID NO: 10 - DNA, 17 bp, Artificial Sequence
  Synthetic
gtcactctgcgagcggc

SEQ ID NO: 11 - DNA, 19 bp, Artificial Sequence
  Synthetic
gctctgattccacgcggct

SEQ ID NO: 12 - DNA, 28 bp, Artificial Sequence
  Synthetic
tctgattccacgcggctcgctctaactt

(19) United States
(12) Patent Application Publication
(10) Pub. No.: US 2016/0312266 A1
(43) Pub. Date: Oct. 27, 2016

(54) METHODS FOR AUTOMATED CAPTURE AND PURIFICATION OF MULTIPLE NUCLEIC ACID TARGETS FROM STOOL SAMPLES

(71) Applicants: YIDING LIU, TWINSBURG, OH (US); BAOCHUAN GUO, SOLON, OH (US)

(72) Inventors: YIDING LIU, TWINSBURG, OH (US); BAOCHUAN GUO, SOLON, OH (US)

(21) Appl. No.: 14/809,487

(22) Filed: Jul. 27, 2015

Related U.S. Application Data
(60) Provisional application No. 62/030,027, filed on Jul. 28, 2014.

(51) Int. Cl. C12Q 1/68

(52) U.S. Cl. CPC C12Q 1/6806

(57) ABSTRACT

Provided herein is technology relating to automated capture and purify nucleic acids from biological samples. In particular, the technology relates to methods for automated capturing, enriching, and purifying multiple nucleic acid targets from human stool samples.

### CROSS-REFERENCE TO RELATED APPLICATION

The present application claims priority to and the benefit of U.S. provisional patent application No. 62/030,027 filed Jul. 28, 2014, the entire disclosure of which is incorporated herein by reference.

FIELD OF INVENTION

Provided herein is technology relating to automated isolation and purification of nucleic acids from biological samples. In particular, the technology relates to methods for automated isolating purified and highly concentrated nucleic acid targets from biological samples, especially from human fecal samples.

### BACKGROUND OF THE INVENTION

Retrieving purified nucleic acid targets from stool samples is essential to fecal nucleic acid testing. Conventional nucleic acids extraction methods are having difficulties for this application because they extract total nucleic acids. Human feces naturally contain a large amount of bacteria and they in turn generate huge amounts of bacterial nucleic acids. Human DNA that is the most commonly used nucleic acid biomarkers may constitute less than 0.1% of total stool nucleic acids (Ahlquist et al, 2000). The presence of large amounts of bacterial nucleic acids can inhibit polymerase chain reaction (PCR) and therefore limits the amount of human DNA that can be loaded into PCR. On the other hand, the microbe composition is so complex that when certain pathogen nucleic acids are of interest, the vast amount of other irrelevant bacterial nucleic acids co-extracted will post challenges for a sensitive and specific assay.

The problem is further compounded when a panel of more than one nucleic acid sequences needed to be detected. Detecting rare events, such as disease related mutations and methylation states of certain genes, requires preparation of highly purified and highly concentrated DNA from biological samples like stool so that enough target sequences can be loaded into assays like PCR. Detecting a panel of multiple rare events like mutations and methylation states of genes can increase the assay sensitivity and specificity, but also post stricter requirements on the amount of disease related DNA loaded into PCR and the purity of the DNA preparation (US 2012/0288867 A1).

Removal of irrelevant interference nucleic acids along with other inhibitors and impurities while selectively retaining target nucleic acid sequences could not be done with conventional methods because they extract total nucleic acids. As such, sequence specific capture (SSC) becomes the method of choice to retrieve target nucleic acid sequences from samples such as stool for it can selectively capture target nucleic acid sequences. SSC is based on sequence specific hybridization between a target sequence and an oligonucleotide capture probe complementary at least to a part of that target sequence. When SSC is performed with probe conjugated magnetic beads, in which captured probes are pre-immobilized onto magnetic beads, the magnetic beads containing capture probes are incubated with a sample to capture target sequences (Jungell-Nortamo et al., 1988). Then, the beads are collected and washed to remove unwanted species. Finally, captured target sequence is eluted to yield purified nucleic acid preparation or used directly along with magnetic beads for the following analysis processes.

However, deploying sequence specific capture in stool DNA testing for diagnostic purpose still encounters multiple problems. First, probe conjugated magnetic beads must be suspended in solution to ensure effective hybridization between target sequences and the probes immobilized onto beads. Prior arts mainly utilize rotating a hybridization container (or tube) that contains a nucleic acid sample, probe conjugated magnetic beads and hybridization buffer for this purpose, which often cannot distribute beads evenly in solution and is difficult to control when the sample volume is large (US 2012/0288867 A1). This will in turn negatively affect the capture efficiency. Second, when handling large volumes of samples, i.e. 10 mL or more in a 50 mL centrifuge tube, prior art requires the use of large, specially designed magnets to ensure effective recovery of magnetic beads. This is because stool solutions are highly viscous, impeding the recovery of magnetic beads. Third, after collecting the beads, prior art methods transfer viscous stool solution to another tube so that the collected beads can be washed and DNA captured can be eluted from the beads (US 2012/0262260 A1). This step is particularly problematic when multiple sequences are sequentially captured by one by one from a large volume of stool solution, requiring transferring more than 10 milliliter of stool solution from tube to tube and instrument to instrument for multiple times. This is a highly labor-intensive process even with large, specially designed instruments, and thus is not practical for a high throughput operation needed for clinical applications.

The current invention provided a highly efficient automated method for sequence-specific capture (SSC) of multiple target nucleic acid sequences from biological sample such as human feces. It is specifically useful in nucleic acids extraction and purification from a large volume of stool solution to detect low-abundance nucleic acid biomarkers in a large background of interference nucleic acids and impurities.

### BRIEF DESCRIPTION OF DRAWINGS

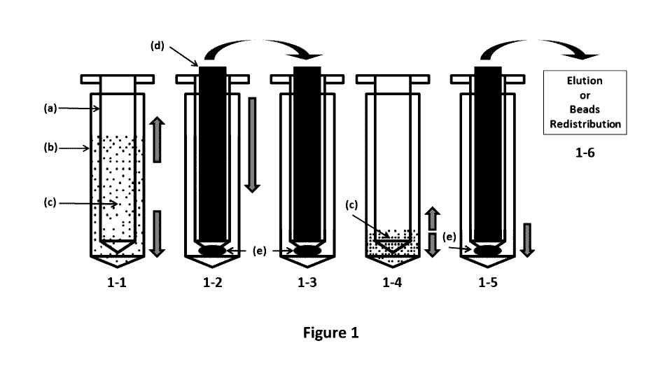

FIG. 1A schematic demonstration of automated sequence specific capture method using probe conjugated magnetic beads for nucleic acid targets capture and purification.
1-1 Automated hybridization between nucleic acid targets in stool sample and probe conjugated magnetic beads in a well with mechanical stirring
   (a) One of the tips on the tip comb
   (b) One of the deep wells on the plate
   (c) Probe conjugated magnetic beads suspended in sample solution (or buffer in following steps)
1-2 Automated beads collection with magnet.
   (d) Part of the magnet
   (e) Magnetic beads collected on the tip comb
1-3 Automated beads transfer to a new well containing wash buffer.
1-4 Automated beads wash with mechanical stirring.

Note: multiple washes are performed with a series of wash buffers.
1-5 Automated beads collection with magnet.
1-6 Automated beads transfer to a new well containing either elution buffer for nucleic acids elution, or beads buffer for beads redistribution (use directly for analysis).

### DETAILED DESCRIPTION OF INVENTION

Nucleic acids extraction and purification is an essential step of many nucleic acid-based diagnostic assays. Conventional methods depend on total nucleic acids extraction and are designed to extract and purify nucleic acids from a small sample, i.e. less than 250 mg stool. The vast majority of total stool nucleic acids come from bacteria, only less than 0.1% of which is actually human DNA, the most commonly used nucleic acid biomarkers for human diseases. Co-isolation of total nucleic acids will not only limit the capacity of the method, but also reduced the efficiency of purification. To minimize inhibitory effects on following PCR based assays, conventional methods often utilize alcohol precipitation, protease digestion and other purification approaches to remove inhibitors and impurities before or after DNA isolation process, which is not effective when dealing with a large stool sample. Moreover, a large background of interference nucleic acids can inhibit PCR based assays even when the nucleic acids preparation is sufficiently purified.

As stool DNA testing is emerging as a promising molecular approach to cancer screening and diagnosis, the problems associated with conventional nucleic acid isolation methods make them more and more unacceptable for the purpose, especially when detecting rare events like mutations and methylation states of genes in stool DNA. Detecting such rare events requires highly concentrated DNA being loaded into PCR assays to ensure enough targeted DNA sequences present for detection. The DNA preparation also needs to be sufficiently purified with such high concentration, but without inhibitory effects. Furthermore, to enhance the sensitivity and specificity of diagnosis, a panel of nucleic acid based biomarkers are often detected in one assay. To prepare enough DNA to detect multiple biomarkers, DNA preparation from 1 gram or more stool sample is often needed, which is beyond the capacity of the conventional DNA extraction methods.

In prior arts, sequence-specific capture (SSC) is introduced for capturing targeted nucleic acid strands while removing interference nucleic acids and inhibitors. SSC becomes the method of choice to retrieve nucleic acids from stool because it can selectively capture target sequences by sequence specific hybridization between a target sequence and oligonucleotide capture probes complementary at least to a part of that target sequence. In prior arts, the probe conjugated magnetic beads, on which the capture probes are pre-immobilized, are first used to capture target sequences from a large sample solution, followed by washing the beads (further purification). Finally, captured sequences are eluted from the beads in a small volume with high concentration.

For stool DNA testing, the probe conjugated magnetic beads are incubated with a denatured stool sample to capture target sequences by hybridization. Then, the beads are collected and washed to remove unwanted species such as interference DNA (non-complementary to probes), inhibitors, and other impurities. In prior art, the hybridization step is performed by rotating or shaking the tubes that contain a sample solution, hybridization buffer and magnetic beads to create a constant movement of the beads to achieve higher hybridization efficiency. However, magnetic beads tend to aggregate during this hybridization process, thus adversely affects capture of target sequence.

On the other hand, the SSC process in prior art is often performed multiple times when multiple target sequences are being captured. In general, after one target sequence is captured, magnetic beads along with the sequences captured by them are collected on the inner surface of the tube with a magnet (this is called the fixing-beads-moving-solution approach (US 2012/0288867 A1), while stool solution is transferred into a new tube for capturing another target sequence. In other words, in this fixing-beads-moving solution approach, the solution is transferred, while the beads stay in the original tube). The entire process of performing hybridization, collecting beads, transferring stool sample solution is repeated until all target sequences are captured sequentially one by one.

The previously reported methods posted many problems that greatly impede their utilization in clinical setting. First, pre-purification of stool can result in a very large volume of stool solution. This requires specially designed bulky instruments for sample handling with human intervention, including a large magnet unit for beads collection. As a result, stool samples have to be handled in a centralized facility instead of in regular clinical labs in order to make this process work.

Second, the fixing-beads-moving-solution strategy does not work well when used to process a large sample, especially to process highly viscous samples like stool solution. Third, sequential capture of multiple sequences requires multiple steps of transferring large volumes of stool solution between tubes, as well as multiple steps of transferring large volumes of stool solution between instruments, which is highly labor intensive and prone to human errors or contaminations.

Current invention provided an automated method to solve aforementioned problems encountered in stool DNA testing. In some embodiments, a specially treated stool sample is used. The treatment step removed and blocked inhibitors and impurities from being carried into DNA preparations as well as keeping sample volume minimum, for example, 4 mL per 1 gram of stool. In some embodiments, the treatment will also clarify stool sample solution so that magnetic beads will not aggregate during the SSC process. In some embodiments, the stool sample containing target DNA sequences is denatured for sequence specific capture. In some embodiments, the stool sample is denatured chemically or by heating for sequence specific capture.

As shown in FIG. 1, in some embodiments, the current invention uses a series of deep wells or plates for the purposes of sequence specific capture (with oligonucleotide probe conjugated magnetic beads), multiple steps of nucleic acids purification (beads wash), and nucleic acids elution or beads redistribution (re-suspension) in solution for direct use in the following analysis assays. In some embodiments, only magnetic beads (along with target nucleic acid sequences captured by them) are transferred between wells/plates, and there are no steps of transferring sample solution or buffer during the nucleic acids extraction and purification process. Please note that the current invention will not be limited to certain types of deep well plates. The type of the plates and the matching tips and magnet is depending on the design of the specific automated instrument platform only.

In some embodiments, denatured stool samples are loaded into wells/plates containing probe conjugated magnetic beads. In some embodiments, a tip comb with multiple tips is used. In some embodiments, the tips on tip comb are used to stir the solution without magnet inserted. In some embodiments, the tips without magnet inserted move in a specified direction at a specific speed to mix probe conjugated beads thoroughly with sample solutions. In some embodiments, the tips without magnet inserted move at another specific speed to help probe conjugated beads suspend evenly in the sample solution, wherein the speed is fast enough to prevent beads aggregation, but slow enough to facilitate hybridization based sequence specific capture. In some embodiments, the tips without magnet inserted can move in more than one direction to help suspend probe conjugated magnetic beads evenly in the sample solution during hybridization. In some embodiments, the volume of the stool sample solution processed is up to 4 mL. In some embodiments, the volume of the stool sample solution processed is more than 4 mL. In some embodiments, the current invention is not limited to certain volume of sample. The sample volume processed is only limited by the instrument used and can be scaled to meet specific needs.

In some embodiments, after sequence specific capture, tips are lifted above the sample solution surface level and magnet is inserted in the tips. In some embodiments, beads collection is performed with tips (magnet inserted) moving into the sample solution, and repeated until all magnetic beads are collected on the lower end of tips. In some embodiments, fully collecting all beads from viscous stool samples takes up to 15 minutes with up to 15 repeats automatically. In some embodiments, the beads collection process could be further adjusted for optimum results depending on the sample type. In some embodiments, magnetic beads along with target sequences captured by them are collected and transferred to another well/plate containing wash buffers. In some embodiments, wash buffers contain salts to keep hybrids stable. In some embodiments, wash buffers contains surfactants to wash off inhibitors and impurities such as, but not limited to, bile acids, polyphenols, fatty acids, proteins and polysaccharides. In some embodiments, wash buffers contain pH stabilizers. In some embodiments, beads wash is repeated multiple times to achieve optimal purification. In some embodiments, the number of beads wash steps could be adjusted to fit specific needs. In some embodiments, the preferred wash buffer volume is in a range from 50 μL to 2000 μL. In some embodiments, the volume of wash buffers can be varied and optimized for different samples or plates. In some embodiments, the composition of wash buffers can be varied and optimized for different washing steps.

In some embodiments, magnet is inserted into the tips during some of the washing steps so that the beads will not be suspended in the washing buffer during wash. In some embodiments, magnetic beads along with their captured target sequences are collected and transferred into another well/plate containing either elution buffer or beads re-suspension buffer. In some embodiments, magnet is removed during the elution or bead re-suspension process. In some embodiments, captured targeted sequences are eluted by heating. In some embodiments, the preferred temperature for elution is in a range of 60° C. to 90° C., but higher temperature can be used. In some embodiments, the preferred elution temperature is about 70° C. The eluted DNA is ready for assays like PCR. Moreover, the eluted DNA preparation is highly concentrated and sufficiently purified. With the current invention, we can load a sample volume as much as 40% of total PCR reaction volume without inhibitory effects. In some embodiments, magnetic beads along with their captured target sequences are re-suspended in said beads re-suspension buffer, which can be used directly in following analysis assays.

With current invention, the whole nucleic acid extraction and purification process can be fully automated after denaturing of nucleic acids in stool samples. Magnetic beads are mixed, suspended, collected, and transferred automatically by one instrument. As a result, no further human intervention is needed until the captured target sequences are eluted or the beads are re-suspended in solution along with their captured target sequences. The DNA preparation is ready to use in following analysis assay. It is preferred that a panel of multiple target nucleic acids sequences is captured simultaneously in one operation. In general, probe conjugated magnetic beads are first prepared individually with one type of capture probes that are complementary to one specific target sequence, then individually prepared beads are mixed to form a multiplexed beads mixture, with which the whole panel of target nucleic acid sequences can be captured simultaneously. In another methods, probe conjugated beads can also be prepared in a multiplexed manner, wherein all oligonucleotide probes for capturing a panel of multiple targeted nucleic acid sequences are simultaneously conjugated onto the magnetic beads, wherein each individual bead can contain multiple different probes to capture multiple sequences. Furthermore, the individually prepared single-probe conjugated magnetic beads can be used with current invention, so that multiple target nucleic acid sequences can be captured sequentially (one by one).

The current invention is most useful for stool DNA testing. Stool samples are very complex and contain vast amounts of inhibitors and impurities. Stool sample solution is highly viscous and hard to process when its volume is more than 1 milliliter. Instead of removing and transferring a large volume of viscous stool solution between containers and instruments, current invention deploys a moving-beads technology, wherein magnetic beads are mixed, suspended, collected, and transferred by a single instrument automatically and no solutions or buffers are transferred. Multiple samples can be processed in parallel on the same instrument thus the throughput of the method is highly improved for clinical applications. Furthermore, the current invention employs mechanical stirring to improve the hybridization efficiency. As a result, stool samples with different viscosity can be processed with minimum variations in the quantity and purity of stool DNA captured.

### Experiments

### EXAMPLE 1

In this experiment, we test sequence specific capture by mechanically stirring (or suspending) probe conjugated beads in stool solution with an automated instrument, and compare it with the prior art tube-rotation method. As aforementioned, suspending probe conjugated beads evenly in the sample solution during hybridization is essential to sequence specific capture. In prior art, this can be achieved by rotating the tube (up and down) containing the sample solution and the beads (called the tube-rotation method). The current invention employed a mechanically stirring method to suspend the beads in stool solution. Experimentally, stool samples collected from 2 volunteers were tested, which were preserved (buffer/stool ratio, 2 mL buffer per 1 gram of stool, v/w) with a preservation buffer formulated by GLC Biotechnology, Inc. A portion of homogenized stool was aliquoted and mixed 1:1 with a stool treatment and hybridization buffer also formulated by GLC Biotechnology, Inc. The mixture was incubated 2 hours under room temperature, and then centrifuged at 10800 g for 20 minutes to precipitate solid particles and impurities. About 4 mL supernatant (equivalent to 1 gram of stool) were used for each sequence specific capture experiment. For each sample, one aliquot was hybridized with the assistance of the tube rotation method as described in prior art, while two aliquots was hybridized with the assistance of our invented mechanical stirring method, which was carried out by an automated instrument. Sequence specific capture was performed after heat denaturing the treated stool sample solution at 95° C. for 10 minutes, following by cooling down it rapidly. 20 μL probe conjugated magnetic beads were used for each aliquot of sample. Thereafter, all beads washing steps were performed manually for all three aliquot samples.

After sequence specific capture and beads washing, captured DNA sequences were eluted in 50 μL tris buffer by heating the beads at 70° C. for 10 minutes on a thermal cycler.

20 mg equivalent captured stool DNA was loaded into a 20 μL quantitative PCR (qPCR or real-time PCR) reaction, and the amount of stool DNA captured was quantified by capturing and analyzing a human beta-actin (ACTB) gene sequence. The PCR condition used is described as below: Precision-melt HRM Master Mix (final concentration 1×, Biorad), primers (final concentration 0.5 μM), and stool DNA (equivalent to 20 mg stool) were mixed and the total PCR volume was adjusted to 20 μL with nuclease free water. Real-time qPCR was performed on Light-cycler 480 II real-time thermal cycler (Roche). The qPCR protocol used is described as below: 95° C. for 10 min, followed by 50 cycles of 95° C. for 20 seconds, 60° C. for 30 seconds, and 72° C. for 45 seconds, followed by a 72° C. extension for 3 minutes. Then a high-resolution-melting (HRM) curve is generated for quality control with the following settings: 95° C. for 1 minute, 60° C. for 1 minute, then a melting curve was obtained from 60° C. to 95° C. with an increment of 0.02° C./second and 25 acquisitions/° C. The final results were analyzed with Light-cycler 480 software provided by Roche.

TABLE 1
The Copy Number of ACTB Sequences Captured
By Mechanically Stirring and Tube-Rotation Methods

| | Stool Sample 1 | Stool Sample 2 |
|---|---|---|
| Tube-Rotation | 440 | 276 |
| Mechanically Stirring Aliquot 1 | 449 | 302 |
| Mechanically Stirring Aliquot 2 | 420 | 338 |

For human beta-actin (ACTB):
Primer
(SEQ ID NO 1)
5′-TTG CTT TTT CCC AGA TGA GC-3′
Primer
(SEQ ID NO 2)
5′-ACA CTC CAA GGC CGC TTT AC-3′
Capture Probe
(SEQ ID NO 3)
5′-CCT TGT CAC ACG AGC CAG TGT TAG TAC CTA CAC
C-3′

Triplet qPCRs were performed for each sample and the results were averaged to minimize PCR variations. The result is shown in Table 1 and the quantity of DNA captured is represented as the copy number of the ACTB strands captured from 20 mg stool. The results clearly demonstrate that the sequence specific capture process achieved by the mechanical stirring method performed well.

### EXAMPLE 2

In this experiment, a test was performed to compare our fully automated sequence specific capture protocol with conventional manual capture protocol. For the fully automated protocol, the steps of hybridization assisted by mechanically stirring, beads collection and beads wash were all performed automatically with an instrumental system without human intervention. For the manual protocol, hybridization was assisted by the tube-rotation method, and the beads collection and wash steps were manually performed. Stool samples collected from 2 other volunteers were tested, which were preserved (buffer/stool ratio, 2 mL buffer per 1 gram of stool, v/w) with a preservation buffer formulated by GLC Biotechnology, Inc. A portion of homogenized stool was aliquoted and mixed 1:1 with a stool treatment and hybridization buffer also formulated by GLC Biotechnology, Inc. The mixture was incubated 2 hours under room temperature, and then centrifuged at 10800 g for 20 minutes to precipitate solid particles and impurities. About 4 mL supernatant (equivalent to 1 gram of stool) were used for each sequence specific capture experiment. For each sample, one aliquot was hybridized with an assistance by the tube rotation method as described in prior art, while two aliquots were hybridized with an assistance with our mechanically stirring method, which was carried out by an automated instrument. Sequence specific capture was performed after heat denaturing the treated stool sample solution at 95° C. for 10 minutes, following by cooling down it rapidly. 20 μL probe conjugated magnetic beads were used for each aliquot of sample.

After sequence specific capture and beads washing, captured sequences were eluted in 200 μL tris buffer by heating the beads at 70° C. for 10 minutes. For the automated method, the same instrument used carried out DNA elution automatically. For manual method, the elution was performed on a thermal cycler and magnetic beads were removed on magnetic rack.

20 mg equivalent captured stool DNA was loaded into a 20 μL quantitative PCR (qPCR) reaction, and the amount of stool DNA captured was quantified by capturing and detecting a human beta-actin (ACTB) gene sequence. The PCR condition is described as below: Precision-melt HRM Master Mix (final concentration 1×, Biorad), primers (final concentration 0.5 μM), and stool DNA (equivalent to 20 mg stool) were mixed and the total PCR volume was adjusted to 20 μL with nuclease free water. Real-time qPCR was performed on Light-cycler 480 II real-time thermal cycler (Roche). The qPCR protocol used is described as below: 95° C. for 10min, followed by 50 cycles of 95° C. for 20 seconds, 60° C. for 30 seconds, and 72° C. for 45 seconds, followed by a 72° C. extension for 3 minutes. And then a high-resolution-melting (HRM) melting curve is generated for quality control with the following settings: 95° C. for 1 minute, 60° C. for 1 minute, then a melting curve is obtained from 60° C. to 95° C. with an increment of 0.02° C./second and 25 acquisitions/° C. The final results are analyzed with Light-cycler 480 software provided by Roche.

For human beta-actin (ACTB):
Primer
(SEQ ID NO 1)
5′-TTG CTT TTT CCC AGA TGA GC-3′
Primer
(SEQ ID NO 2)
5′-ACA CTC CAA GGC CGC TTT AC-3′
Capture Probe
(SEQ ID NO 3)
5′-CCT TGT CAC ACG AGC CAG TGT TAG TAC CTA CAC
C-3′

TABLE 2
Automated SSC Protocol vs. Manual SSC Protocol

| | Stool Sample 3 | Stool Sample 4 |
|---|---|---|
| Manual | 412 | 115 |
| Automated Aliquot 1 | 510 | 152 |
| Automated Aliquot 2 | 466 | 121 |

Triplet qPCRs were performed for each aliquot and the results were averaged to minimize PCR variations. The result is shown in Table 2 and the quantity of DNA is represented as the copy number of the ACTB strands captured from 20 mg stool. The results clearly demonstrate that our fully automated protocol works well when used to capture DNA from stool. With current invention, the automated method is capable of processing 24 stool samples simultaneously, a great improvement in throughput over prior art methods.

### EXAMPLE 3

In this set of experiments, a reproducibility test was performed for our fully automated sequence specific capture method. Stool samples collected from 3 volunteers were tested, which were preserved (buffer/stool ratio, 2 mL buffer per 1 gram of stool, v/w) with a preservation buffer formulated by GLC Biotechnology, Inc. A portion of homogenized stool was aliquoted and mixed 1:1 with a stool treatment and hybridization buffer also formulated by GLC Biotechnology, Inc. The mixture was incubated 2 hours under room temperature, and then centrifuged at 10800 g for 20 minutes to precipitate solid particles and impurities. About 4 mL supernatant (equivalent to 1 gram of stool) were used for each sequence specific capture experiment. For each sample, one aliquot was hybridized with an assistance by the tube rotation method as described in prior art, two aliquots were hybridized with an assistance with our mechanical stirring method, which was carried out by an automated instrument. Sequence specific capture was performed after heat denaturing the treated stool sample solution at 95° C. for 10 minutes, following by cooling down it rapidly. 20 μL probe conjugated magnetic beads were used for each aliquot of sample. Bovine serum albumin was also added to eliminate inhibitors and impurities during sequence specific capture.

After sequence specific capture and beads washing, captured DNA sequences were eluted in 200 μL tris buffer by heating the beads at 70° C. for 10 minutes automatically on the same instrument used for the above steps.

10 mg equivalent of captured stool DNA were loaded in a 20 μL quantitative PCR (qPCR), and the amount of stool DNA captured is quantified by capturing and detecting a human beta-actin (ACTB) gene sequence. The PCR condition is described as below: Precision-melt HRM Master Mix (final concentration 1×, Biorad), primers (final concentration 0.5 μM), and stool DNA (equivalent to 10 mg stool) were mixed and the total PCR volume was adjusted to 20 μL with nuclease free water. Real-time qPCR was performed on Light-cycler 480 II real-time cycler (Roche). The qPCR protocol used is described as below: 95° C. for 10 min, followed by 50 cycles of 95° C. for 20 seconds, 60° C. for 30 seconds, and 72° C. for 45 seconds, followed by a 72° C. extension for 3 minutes. And then a high-resolution-melting (HRM) melting curve is generated for quality control with the following settings: 95° C. for 1 minute, 60° C. for 1 minute, then a melting curve is obtained from 60° C. to 95° C. with an increment of 0.02° C./second and 25 acquisitions/° C. The final results are analyzed with Light-cycler 480 software from Roche.

For human beta-actin (ACTB):
Primer
(SEQ ID NO 1)
5′-TTG CTT TTT CCC AGA TGA GC-3′
Primer
(SEQ ID NO 2)
5′-ACA CTC CAA GGC CGC TTT AC-3′
Capture Probe
(SEQ ID NO 3)
5′-CCT TGT CAC ACG AGC CAG TGT TAG TAC CTA CAC
C-3′

TABLE 3
Reproducibility Test of Our Automated
Sequence Specific Capture Method

| Sample | Repeat | Repeat | Repeat | Standard |
|---|---|---|---|---|
| ID | 1 | 2 | 3 | Deviation |
| 5 | 152 | 113 | 103 | 0.09 |
| 6 | 241 | 209 | 211 | 0.06 |
| 7 | 740 | 713 | 701 | 0.07 |

Triplet qPCRs were performed for each sample and the results were averaged to minimize PCR variations. The result is shown in Table 3 and the quantity of DNA captured is represented as the copy number of ACTB strands captured from 10 mg stool. The results clearly demonstrate that our fully automated sequence specific capture method was highly reproducible over triplet repeating tests.

### EXAMPLE 4

In this set of experiments, a test was performed to study capture multiple sequences and compare the performance of three (3) sequence specific capture approaches (Approach A, B, C, respectively). Stool samples collected from 4 volunteers were tested and each stool sample was preserved (buffer/stool ratio, 2 mL buffer per 1 gram of stool, v/w) with a preservation buffer formulated by GLC Biotechnology, Inc. A portion of homogenized stool was aliquoted and mixed 1:1 with a stool treatment and hybridization buffer also formulated by GLC Biotechnology. Inc. The mixture was incubated for 2 hours at room temperature, and then centrifuged at 10800 g for 20 minutes to precipitate solid particles and impurities. About 4 mL supernatant (equivalent to 1 gram of stool) was used for each sequence specific capture experiment.

For Approach A, probe conjugated magnetic beads were prepared in a multiplexed manner, i.e. 4 types of capture probes were conjugated onto magnetic beads simultaneously, each of which captures one specific sequence. As a result, every bead contained all 4 probes and could capture 4 target sequences at the same time. For Approach B, each type of capture probes was first conjugated to magnetic beads individually. Then, the 4 batches of the magnetic beads, each of which captures one of 4 target sequences, were mixed together for simultaneously capturing 4 target sequences. For Approach C, each type of capture probes was conjugated onto magnetic beads individually. Then, the 4 sequences were captured one by one sequentially using one batch of individually prepared beads a time. In Approach C, 10 μL beads for each target sequence were used (a total of 40 μL for 4 sequences). In Approaches A and B, 40 μL beads were used for simultaneously capturing 4 sequences. All steps of beads washing and transferring were performed automatically on an automated instrument platform. Sequence specific capture was performed after heat denaturing the treated stool sample solution at 95° C. for 10 minutes, following by cooling down it rapidly. Bovine serum albumin was also added to eliminate inhibitors and impurities during sequence specific capture.

After sequence specific capture and beads washing, DNA sequences captured were eluted in 200 μL tris buffer by heating the beads at 70° C. for 10 minutes automatically on the same instrument used for automated hybridization and beads collection and wash. In Approach C, after each sequence specific capture, stool solutions were transferred into a new sets of wells containing a new batch of probe conjugated magnetic beads for capturing another sequence. Approaches A and B resulted in one elution solution containing all 4 target sequences, while Approach C resulted in 4 elution solutions, each of which contained one target sequence. The 4 target sequences included human beta-actin (ACTB, Gene 1) and three other genes (Gene 2, 3, 4, respectively).

10 mg equivalent captured stool DNA were loaded into a 20 μL quantitative PCR (qPCR) reaction, and the amount of stool DNA captured is quantified individually for all 4 target sequences. For Genes 1 and 2, the PCR conditions used were the same and are described as below: Precision-melt HRM Master Mix (final concentration 1×, Biorad), primers (final concentration 0.5 μM), and stool DNA (equivalent to 10 mg stool) are mixed and the total PCR volume is adjusted to 20 μL with nuclease free water. Real-time qPCR is performed on Light-cycler 480 II real-time cycler (Roche). The qPCR protocol is described as below: 95° C. for 10min, followed by 50 cycles of 95° C. for 20 seconds, 60° C. for 30 seconds, and 72° C. for 45 seconds, followed by a 72° C. extension for 3 minutes. And then a high-resolution-melting (HRM) curve is generated for quality control with the following settings: 95° C. for 1 minute, 60° C. for 1 minute, then a melting curve is obtained from 60° C. to 95° C. with an increment of 0.02° C./second and 25 acquisitions/° C. The final results are analyzed with Light-cycler 480 software from Roche.

For Gene 1 (ACTB):
Primer
(SEQ ID NO 1)
5′-TTG CTT TTT CCC AGA TGA GC-3′
Primer
(SEQ ID NO 2)
5′-ACA CTC CAA GGC CGC TTT AC-3′
Capture Probe
(SEQ ID NO 3)
5′-CCT TGT CAC ACG AGC CAG TGT TAG TAC CTA CAC
C-3′
For Gene 2 (KRAS):
Primer
(SEQ ID NO 4)
5′-TAT TTT TAT TAT AAG GCC TGC TGA AAA TGA CT-3′
Primer
(SEQ ID NO 5)
5′-GAA TTA GCT GTA TCG TCA AGG CAC TCT-3′
Capture Probe
(SEQ ID NO 6)
5′-CCA CAA GTT TAT ATT CAG TCA TTT TCA GCA GGC
C-3′

For Genes 3 and 4, the PCR conditions used were the same and are described as below: Precision-melt HRM Master Mix (final concentration 1×, Biorad), primers (final concentration 0.5 μM), and stool DNA (equivalent to 10 mg stool) are mixed and the total PCR volume is adjusted to 20 μL with nuclease free water. Real-time qPCR is performed on Light-cycler 480 II real-time cycler (Roche). The qPCR protocol is described as below: 95° C. for 10min, followed by 50 cycles of 95° C. for 20 seconds, 66° C. for 30 seconds, and 72° C. for 45 seconds, followed by a 72° C. extension for 3 minutes. And then a high-resolution-melting (HRM) curve was generated for quality control with the following settings: 95° C. for 1 minute, 60° C. for 1 minute, then a melting curve is obtained from 60° C. to 95° C. with an increment of 0.02° C./second and 25 acquisitions/° C. The final results are analyzed with Light-cycler 480 software from Roche.

For Gene 3 (ALX4):
Primer
(SEQ ID NO 7)
5′-CTG CGC AAG CCA GGC ATG AA-3′
Primer
(SEQ ID NO 8)
5′-GAG CCC TCC CGA CTC TGC GA-3′
Capture Probe
(SEQ ID NO 9)
5′-TCG CAG TAA GAG ACG CAA GTC TCA GCA TTC ATG-3′
For Gene 4 (PHA3):
Primer
(SEQ ID NO 10)
5′-GTC ACT CTG CGA GCG GC-3′
Primer
(SEQ ID NO 11)
5′-GCT CTG ATT CCA CGC GGC T-3′
Capture Probe
(SEQ ID NO 12)
5′-TCT GAT TCC ACG CGG CTC GCT CTA ACT T-3′

Two qPCRs were performed for each sample and the results were averaged to minimize PCR variations. The result is shown in Table 4 and the quantity of DNA is represented as the copy number of DNA strands captured in 10 mg stool.

TABLE 4
Multiplexed v.s. Sequential Sequence Specific Capture

| | Gene 1 | | | Gene 2 | | |
|---|---|---|---|---|---|---|
| | Ap- | Ap- | Ap- | Ap- | Ap- | Ap- |
| Sample | proach | proach | proach | proach | proach | proach |
| ID | A | B | C | A | B | C |
| 1 | 218 | 290 | 336 | 83 | 92 | 78 |
| 2 | 90 | 131 | 88 | 158 | 181 | 174 |
| 3 | 591 | 714 | 680 | 96 | 95 | 87 |
| 4 | 396 | 605 | 639 | 157 | 140 | 151 |
| | Gene 3 | | | Gene 4 | | |
| | Ap- | Ap- | Ap- | Ap- | Ap- | Ap- |
| Sample | proach | proach | proach | proach | proach | proach |
| ID | A | B | C | A | B | C |
| 1 | 981 | 897 | 1028 | 1290 | 1587 | 921 |
| 2 | 389 | 321 | 317 | 791 | 876 | 833 |
| 3 | 1304 | 1439 | 1448 | 1827 | 2282 | 1622 |
| 4 | 1874 | 1830 | 2283 | 2654 | 3255 | 3105 |

The results demonstrated that multiplexed sequence specific capture methods (Approaches A and B) worked comparatively well with sequential sequence specific captures (Approach C). And more importantly, all three approaches can be performed with the automated platform developed in the current invention.

### CITATION LIST

### Patent Literature

Lidgard et al. US 2012/0288867 A1
Light, II et al. US 2012/0262260 A1

### Non Patent Literature

Ahlquist D A, Skoletsky et al., Colorectal cancer screening by detection of altered human DNA in stool: feasibility of a multitarget assay panel. Gastroenterology 2000; 119:1219-1227.
Jungell-Nortamo A, et al., Nucleic acid sandwich hybridization: enhanced reaction rate with magnetic microparticles as carriers. Mol Cell Probes 1988; 2:281-288.

What is claimed is:

1. Method for isolation and purification of nucleic acid target(s) in stool sample using sequence specific capture, wherein said method comprises steps of:
(a) performing sequence-specific hybridization between said nucleic acid targets in said stool sample and probe conjugated magnetic beads in a well or plate with mechanical stirring created by a tip comb to capture said nucleic acid targets in said stool sample; and
(b) collecting said magnetic beads along with said captured nucleic acid target(s) after said sequence-specific hybridization by using magnet inserted into said tip comb; and
(c) transferring said collected magnetic beads along with said captured nucleic acid target(s) using said magnet inserted into said tip comb to another well or plate containing wash buffer; and
(d) washing said collected magnetic beads along with said captured nucleic acid target(s) in said another well or plate containing said wash buffer with mechanical stirring created by said tip comb; and
(e) steps (b), (c), and (d) are repeated for one or more times; and
(f) collecting said washed magnetic beads along with said captured nucleic acid target(s) using said magnet inserted into said tip comb and transferring said washed magnetic beads along with said captured nucleic acid target(s) to another well or plate.

2. The method of claim 1, wherein said stool sample is a solution.

3. The method of claim 1, wherein said nucleic acid target(s) are DNA, mRNA, MicroRNA, tRNA, or a combination of them.

4. The method of claim 1, wherein said nucleic acid target(s) in said stool sample has been denatured.

5. The method of claim 1, wherein said mechanical stirring is created by the vertical, horizontal, or any movement of said tip comb.

6. The method of claim 1, wherein said magnet is dissociated from said tip comb during said sequence-specific hybridization with said mechanical stirring.

7. The method of claim 1, wherein said magnet is dissociated from said tip comb during said beads wash with said mechanical stirring.

8. The method of claim 1, wherein said nucleic acid target(s) is single or multiple sequences.

9. The method of claim 8, wherein said multiple sequences are captured simultaneously, captured one by one sequentially, or captured batch by batch sequentially.

10. Method for automated isolation and purification of target DNA sequences(s) in stool sample using sequence specific capture, wherein said method comprises steps of
(a) performing sequence-specific hybridization between denatured target DNA sequences in said stool sample and probe conjugated magnetic beads in a well or plate with mechanical stirring created by a tip comb to capture said target DNA sequences; and
(b) collecting said magnetic beads along with said captured said target DNA sequence(s) after said sequence-specific hybridization by using magnet inserted into said tip comb; and
(c) transferring said collected magnetic beads along with said captured target DNA sequences using said magnet inserted into said tip comb to another well or plate containing wash buffer; and
(d) washing said collected magnetic beads along with said captured target DNA sequences in said another well or plate containing said wash buffer with mechanical stirring created by said tip comb; and
(e) steps (b), (c), and (d) are repeated for one or more times; and
(f) collecting said washed magnetic beads along with said captured target DNA sequence(s) using said magnet inserted into said tip comb and transferring said washed magnetic beads along with said captured target stool sequences to another well or plate.

11. The method of claim 10, wherein said stool sample is a solution.

12. The method of claim 10, wherein said mechanical stirring is created by the vertical, horizontal, or any movement of said tip comb.

13. The method of claim 10, wherein said magnet is dissociated from said tip comb during said hybridization with said mechanical stirring.

14. The method of claim 10, wherein said magnet is dissociated from said tip comb during said beads wash with said mechanical stirring.

15. The method of claim 10, wherein said target DNA sequence(s) is single or multiple sequences.

16. The method of claim 10, wherein said multiple sequences are captured simultaneously, captured one by one sequentially, or captured batch by batch sequentially.

17. The method of claim 10, further comprises eluting said captured target DNA sequence(s) from said washed magnetic beads in said another well or plate containing elution buffer with mechanical stirring created by said tip comb, and removing said magnetic beads after said elution using said tip comb with said magnet inserted in from said elution buffer.

18. The method of claim 10, further comprises suspending said washed said magnetic beads along with said captured target DNA sequence(s) in said another well or plate containing a buffer by mechanical stirring created by said tip comb

19. The method of claim 10, wherein multiple said stool samples are simultaneously processed using one system.
